(19) United States
(12) Patent Application Publication
(10) Pub. No.: US 2014/0314827 A1
(43) Pub. Date: Oct. 23, 2014

(54) Antimicrobial compositions and uses therefor

(71) Applicant: Steven A. Melman, Boca Raton, FL (US)

(72) Inventor: Steven A. Melman, Boca Raton, FL (US)

(21) Appl. No.: 14/120,038

(22) Filed: Apr. 17, 2014

Related U.S. Application Data
(60) Provisional application No. 61/854,151, filed on Apr. 18, 2013.

(51) Int. Cl. A61K 31/155; A01K 13/00; A61K 45/06; A23K 1/17; A61K 31/198; A61K 31/133

(52) U.S. Cl. CPC A61K 31/155; A61K 31/198; A61K 31/133; A61K 45/06; A23K 1/17; A01K 13/001
USPC 424/442; 514/566; 514/192; 514/154; 514/2.3; 514/570; 514/567; 514/226.5; 119/601

(57) ABSTRACT

Described herein are compositions comprising at least one buffering agent, at least one chelating agent and at least one antimicrobial agent, which can be used for treating and/or preventing diseases or disorders in a variety of animals. In certain embodiments, in particular, the compositions can be used for treating and/or preventing ophthalmic and oral cavity diseases or disorders in dogs and cats and oropharyngeal and/or guttural pouch diseases or disorders in equines. In certain embodiments, the compositions can also be used for treating ophthalmic or oral cavity diseases or disorders in humans.

### CROSS REFERENCE TO RELATED APPLICATIONS

This application claims the benefit of priority from U.S. Provisional Patent Application No. 61/854,151 filed Apr. 18, 2013.

### FIELD OF THE INVENTION

The various embodiments of the invention described herein are directed towards antimicrobial compositions and methods for using such compositions in treating certain animal disorders. In particular, the compositions are used for treating ophthalmic and oral cavity related disorders, in particular odontostomatological pathologies, in a variety of animals, as well as treating oropharyngeal and/or guttural pouch disorders in equines. The compositions described comprise at least one pH buffering agent, at least one chelating agent and at least one antimicrobial agent. The compositions may further comprise at least one antibiotic agent and/or at least one anti-inflammatory agent.

### BACKGROUND

Otic compositions comprising ethylenediamine-tetraacetic acid (hereinafter “EDTA”) and tromethamine or TRIS(hydroxymethyl)aminomethane (“TRIS”) are known. See U.S. Pat. No. 6,538,155. Compositions comprising combinations of EDTA and TRIS are known. Such compositions (hereinafter “EDTA-TRIS compositions”) are useful as antimicrobial agents when used alone or in combination with other antimicrobial agents, such as antibiotics. Synergistic activity was observed when EDTA-TRIS compositions plus amikacin and EDTA-TRIS compositions plus neomycin were tested against Staphylococcus intermedius, Proteus mirabilis, Pseudomonas aeruginosa and Escherichia coli. Sparks et al, “Antimicrobial effect of combinations of EDTA-TRIS and amikacin or neomycin on the microorganisms associated with otitis externa in dogs”, Vet. Res. Commun., 1994, Vol. 18 (4), pp. 241-9. There remains, however, a need for therapeutically effective compositions comprising at least one antimicrobial, in particular, at least one antiseptic for treating other animal disorders, which include disorders of the eye and oral cavity in a variety of animals, as well as disorders of the oropharyngeal and/or guttural pouch in equines.

Tear staining is a common problem and concern of pet owners. Excessive tears (epiphora) drain down the pet's face and this chronic exposure to moisture can cause skin irritation, infection, odor, and excessive fur pigmentation. The fur becomes stained reddish brown due to tear pigments called porphyrins. This is especially a problem in pets with white coats (Maltese, Bichon Frise, white-coated Poodle or any white-coated cat) and pets with very small tear ducts, and/or large eyes and short muzzles, all of which tends to allow more eye irritation and less drainage (Cavalier King Charles Spaniel, Persian Cats). It can be caused by excessive tear production, insufficient tear drainage, or a combination of both. Epiphora can be acute or chronic. When epiphora becomes chronic, the constant moisture around the eyes results in skin irritation and creates a fertile breeding ground for bacteria and yeast. Over time, red staining around the eyes is seen due to accumulation of a pigment called porphyrin, which is found in tears. Porphyrins are a group of organic compounds of which many occur in nature. One of the best-known porphyrins is heme, the pigment in red blood cells. Heme is a cofactor of the protein hemoglobin.

A heme (American English) or haem (British English) is a prosthetic group that consists of an iron atom contained in the center of a large heterocyclic organic ring called a porphyrin. Not all porphyrins contain iron, but a substantial fraction of porphyrin-containing metalloproteins have heme as their prosthetic group; these are known as hemoproteins. Hemes are most commonly recognized in their presence as components of hemoglobin but they are also components of a number of other hemoproteins.

When red blood cells are naturally broken down by the body, a chemical substance known as porphyrin is left in the bloodstream. Porphyrins are excreted primarily through bile and the intestinal tract, but in dogs a significant amount of porphyrin is excreted through tears, saliva and also urine. Porphyrin is an iron-containing substance, and staining by porphyrin is difficult to remove.

Staining occurs when pigmentation from a substance penetrates the hair cuticle and is deposited within the cortex of the hair shaft(s). Since the detergent surfactant in a shampoo works on the surface of the hair cuticle, the stain is not removed by shampooing. The hair has in fact been dyed. Dealing with staining is a challenge, often requiring trying various approaches. Daily removal of offending substances such as tears, saliva or urine is recommended.

The chemistry of some dogs' saliva can cause staining, especially if they lick themselves. Hypersalivation (also called ptyalism and sialorrhea) can cause staining of the fur of dog and can occur in response to mouth pain caused by periodontal disease, abscessed teeth, and stomatitis. Accordingly, there is a need for a simple and effective means of treating and/or preventing tear and/or salivary stains in animals, particularly dogs.

Periodontal disease is one of the most common problems seen in veterinary practice. It occurs in two forms: (1) gingivitis, a reversible inflammation of the gums, and (2) periodontitis, an inflammation of the deeper structures supporting the teeth. Gingivitis develops when bacteria build up between the teeth and gums, leading to irritation, inflammation, and bleeding. In a animals with gingivitis, rough dental calculus builds up in an irregular fashion along the gum line, producing points at which the gum is forced away from the teeth. This creates small pockets that trap food and bacteria. In time, the gums become infected.

Dental calculus (also called tartar) is composed of calcium salts, food particles, bacteria, and other organic material. It is yellow-brown and soft when first deposited. At the soft stage it is called plaque. The plaque quickly hardens into calculus. Calculus collects on all tooth surfaces, but is found in the greatest amounts on the cheek side of the upper premolars and molars. This buildup of calculus on the teeth is the primary cause of gum inflammation. This occurs to some extent in all dogs over the age of 2. Certain breeds, such as Poodles, and smaller dogs seem to form calculus more readily.

Current treatment for gingivitis is directed towards professionally cleaning, scaling, and polishing the animal's teeth to remove all plaque and calculus. Many veterinarians now use ultrasonic dental units, similar to the ones used on humans, for cleaning dogs' teeth. The cleaning should then be followed with a regular regimen of home oral care, which includes regular brushing of the animal's teeth.

Periodontitis develops as a continuation of gingivitis. The teeth are held in their bony sockets by a substance called cementum and a specialized connective tissue called the periodontal membrane. As the gum infection attacks the cementum and periodontal membrane, the roots become infected, the teeth begin to loosen, and eventually they detach. A root abscess can rupture into the maxillary sinus or nasal cavity, producing a purulent unilateral nasal discharge, an oral-nasal fistula or a swelling below the eye.

Current treatment for periodontitis includes professionally cleaning the teeth, as described for gingivitis. Severe infections may necessitate removing a portion of the diseased gum (a procedure called gingivectomy). Antibiotics can be given for a period of time, depending on the severity of the disease. Aftercare at home involves rinsing the animal's mouth with a chlorhexidine solution (Peridex® [0.12% chlorhexidine gluconate, water, alcohol, glycerine, PEG-40 sorbitan diisostearate, flavour, saccharin sodium, and FD&C Blue No. 1 Dye] or Nolvadent® [0.1% Chlorhexidine Acetate formulated with special surfactant, peppermint flavored base, FD&C Red 40 & Red 33; 6% Ethyl Alcohol by volume]) once or twice a day. A product called Stomadhex® may also prove to be an effective substitute for the aftercare just described. Stomadhex® is a small adhesive patch that sticks to mucous membranes. The patch is applied to the inside surface of the upper lip. It stays in place for several hours and slowly releases chlorhexidine and a vitamin called nicotinamide that promotes oral hygiene. The sustained release delivery agent helps to prevent dental plaque and tartar and aids in controlling bad breath. The patch is applied daily for 10 days following a dental procedure, or as recommended by a veterinarian. In the veterinary field, the use of compositions based on chlorhexidine, while known in the state of the art for the treatment of odontostomatological pathologies of animals, has various drawbacks, mainly associated with the high dosage that must be administered to animals in order to obtain significant benefits. Accordingly, there is a need for a simple and effective means for treating and or preventing disorders of the oral cavity, particularly odontostomatological pathologies of animals.

Strangles is a highly contagious and serious infection of horses and other equids caused by the bacterium, Streptococcus equi. The disease is characterized by severe inflammation of the mucosa of the head and throat, with extensive swelling and often rupture of the lymph nodes, which produces large amounts of thick, creamy pus.

Strangles is caused by Streptococcus equi subspecies equi, better known as Streptococcus equi (S. equi). Although the organism is not very robust, the infection is highly contagious.

Classic strangles is a severe infection that can be fatal, usually because of a variety of complications that occur. The main and often fatal complications of strangles are: bastard strangles, which describes the dissemination of infection to unusual sites other than the lymph nodes draining the throat and purpura haemorrhagica, which is an immune-mediated acute inflammation of peripheral blood vessels. Minor, non-fatal complications include: post strangles myocarditis (inflammation of heart muscle), purulent cellulitis (inflammation of the subcutaneous tissue), laryngeal hemiplegia, which involves paralysis of the throat muscles, anaemia (low red blood cell count), and guttural pouch empyema (filled with pus), which may be concurrent with classic strangles, or follow in the immediate convalescent period. A guttural pouch is an air-filled out-pouching of the auditory or Eustachian tube. While all mammals have auditory tubes, not all have these guttural pouches. Horses, mules, and donkeys have the largest, with one pouch lying on each side of the back of the throat and diseases that occur in this region can be quite severe, and can have deadly consequences. Guttural pouches are present only in equidae and are situated between the base of the cranium dorsally and the pharynx ventrally. The guttural pouch is a two-chambered space separated by the stylohyoid bone, and can hold roughly 20 ounces of fluid or air. The medial or innermost chamber has several important blood vessels and nerves located on its surface. These nerves are responsible for various crucial body functions, such as breathing, swallowing, and chewing. The lateral or outermost chamber is associated with more nerves and contains the internal carotid artery and the maxillary-facial vein. Proximity of the guttural pouches and the nerves, arteries, and veins associated with them also exposes these critical structures to infection and damage.

With each swallow, air enters or leaves the guttural pouch of equines. This means that any bacteria, fungi, or other infectious agents inhaled or ingested by the equine have ready access to both pouches. These potentially disease-causing particles can enter the pouches and usually become trapped in the mucus that lines these structures. Most of the time, the equine's immune system successfully destroys these agents, but bacteria or fungi occasionally survive and continue to grow by invading the lining of the pouches.

Persistent infection in the guttural pouch may lead to inspissation (drying) of pus and, in some cases, the formation of a solid, stone-like, concretion called a chondroid. Animals that have persistent infection of the guttural pouches become carriers, the major source of infection to spark outbreaks in susceptible horses with which they are mixed.

Current treatment calls for aggressive flushing of the pouch and antibiotic therapy. Specialized catheters are placed in the affected pouch, and large volumes of fluids are repeatedly flushed in and out with significant pressure. If the condition is chronic and severe enough, surgery sometimes is needed to drain the pouch. Frequent use of antibiotics however can lead to antibiotic resistance. There thus remains a need for a simple and effective treatment of oropharyngeal and/or guttural pouch disorders in equines.

### SUMMARY OF THE INVENTION

The various embodiments of the invention described herein are directed to compositions, and methods of their use in treating and/or preventing certain ophthalmic and oral cavity disorders, particularly odontostomological pathologies, in animals and humans. In certain embodiments the compositions are suitably used for flushing or therapeutically irrigating (lavage) infected oropharyngeal area and/or guttural pouches of equines. The compositions described herein are non-toxic, non-irritating, isotonic, possess antimicrobial properties, and have a pH that is compatible with the body organ or area being treated.

In some embodiments the compositions, preparations, and methods described herein may be used with any animal tissue, preferably any mammalian tissues including, but not limited to, human, non-human primate, equine, bovine, ovine, porcine, canine and feline tissues.

In certain embodiments, the invention provides for compositions with at least one buffering agent, at least one metal ion chelating agent, and at least one antimicrobial and optionally at least one anti-inflammatory and/or at least one disinfectant.

In some embodiments, the buffering agent is selected from the group consisting of citrate buffering agent, phosphate buffering agent, borate buffering agent, tris(hydroxymethyl)aminomethane (TRIS) buffering agent, sodium bicarbonate, N,N′-bis(2-hydroxyethyl)glycine (BICIN) buffering agent, 2-[bis(2-hydroxyethyl)amino]-2-(hydroxymethyl)-1,3-propanediol (BISTRIS) buffering agent, 2-(cyclohexylamino)ethane-2-sulfonic acid (CHES) buffering agent, N-2-(hydroxyethyl)piperazine-N′-2-ethanesulfonic acid (HEPES) buffering agent, N-(2-hydroxyethyl)piperazine-N′-3-propanesulfonic acid (HEPPS) buffering agent, morpholinoethanesulfonic acid (MES) buffering agent, morpholinopropanesulfonic acid (MOPS) buffering agent, piperazine-N,N′-bis(2-ethanesulfonic acid) (PIPES) buffering agent, N-tris(hydroxymethyl)methyl-2-aminoethanesulfonic acid (TES) buffering agent, N-tris(hydroxymethyl)methyl-3-aminopropanesulfonic acid (TAPS) buffering agent, and N-tris(hydroxymethyl)methylglycine (TRICINE) buffering agent, and combinations thereof.

In at least one embodiment, the metal ion chelating agent is selected from the group consisting of citric acid, phosphates, the di-, tri- and tetra-sodium salts of ethylene diamine tetraacetic acid (EDTA), ethylene glycol-bis-(b-aminoethylether)-N,N,N′,N′-tetraacetic acid (EGTA); 1,2-bis(2-aminophenoxy)ethane-N,N,N′,N′-tetraacetic acid (BAPTA); ethylene-N,N′-diglycine (EDDA); 2,2′-(ethylendiimino)-dibutyric acid (EBDA); lauroyl EDTA; dilauroyl EDTA, triethylene tetramine dihydrochioride (TRIEN), diethylenetriamin-pentaacetic acid (DPTA), triethylenetetramine hexaacetic acid (TTG), deferoxamine, Dimercaprol, zinc citrate, penicilamine succimer, Editronate, sodium hexmetaphosphate and edetate calcium disodium and combinations thereof.

In at least one embodiment, the metal ion chelating agent is the di-, tri- or tetra-sodium salt of ethylene diamine tetraacetic acid (EDTA). In at least one embodiment, the metal ion chelating agent is the di-sodium salt of EDTA.

In some embodiments, the TRIS-EDTA is present at a concentration of from between 5% (wt/wt) to 100% (wt/wt). In some embodiments, the TRIS-EDTA is from between 25% (wt/wt) to 85% (wt/wt). In some embodiments, the TRIS-EDTA is present from between 50% (wt/wt) to 65% (wt/wt).

In some embodiments, the TRIS-EDTA buffer has a pH ranging from between 5.5 to 8.5. In some embodiments, the TRIS-EDTA buffer has a pH ranging from between 6.0 to 8.0. In some embodiments, the TRIS-EDTA buffer has a pH ranging from between 6.5 to 7.5. In some embodiments, the TRIS-EDTA buffer has a neutral pH.

The anti-microbial agents that can be used in the compositions are well known in the art and can be selected from the group consisting of at least one antifungal agent, at least one antiseptic agent, at least one antibiotic agent, at least one antiviral agent, at least one antiparasitic agent and combinations thereof.

In some embodiments, non-limiting examples of the at least one antifungal agent includes butenafine, clotrimazole, ketoconazole, miconazole, nystatin, terbinafine, ciclopirox, econazole, luliconazole, naftifine, oxiconazole, and combinations thereof.

In some embodiments, non-limiting examples of the at least one antiseptic agent includes triclosan; iodoform; quaternary ammonium compounds, such as for example, benzalkonium chloride, cetyl trimethylammonium bromide, cetylpyridinium chloride, and benzethonium chloride; and biguanides, such as PHMB, N-alkyl-2-pyrrolidone, chiorhexidine, polyquaternium-1, hexetidine, bronopol, alexidine and combinations thereof.

In some embodiments, the at least one antimicrobial agent includes at least one antiparasitic agent. Non-limiting examples of the at least one antiparasitic agent include antinematodes, anticestodes, antitrematodes, antoamoebics, antiprotozoals and combinations thereof. Non-limiting examples of antinematodes include mebendazole, pyrantel pamoate, thiabendazole, diethylcarbamazine, ivermectin and combinations thereof. Non-limiting examples of anticestodes include niclosamide, praziquantel, albendazole and combinations thereof. Non-limiting examples of antitrematodes include praziquantel. Non-limiting examples of antiamoebics include rifampin, amphotericin B and combinations thereof. Non-limiting examples of antiprotozoals include melarsoprol, eflornithine, metronidazole, tinidazole, and miltefosine.

In at least one embodiment, the at least one antimicrobial is at least one antiseptic agent in quantities ranging from between 0.001% (wt/wt) to 5% (wt/wt) of said composition. In some embodiments, the at least one antiseptic agent is present in quantities ranging from between 0.00125% (wt/wt) to 0.5% (wt/wt) of said composition. In certain other embodiments, the at least one antiseptic agent is present in quantities ranging from between 0.00125% (wt/wt) to 0.15% (wt/wt) of said composition. In at least one embodiment, the at least one antiseptic agent is present in quantities ranging from between 0.00125% (wt/wt) to 0.005% (wt/wt) of said composition. In some other embodiments, the at least one antiseptic agent is present in quantities ranging from between 0.00125% (wt/wt) to 0.0025% (wt/wt) of said composition.

In embodiments where the composition is used for treating and/or preventing ophthalmic disorders, the at least one antiseptic agent is present at 0.002% (wt/wt) of said composition.

In embodiments where the composition is used for treating and/or preventing oral cavity disorders, the at least one antiseptic is present at 0.12% (wt/wt) of said composition.

In embodiments where the composition is used for treating and/or preventing oropharyngeal and/or guttural pouch disorders in equines, the at least one antiseptic is present at 0.5% (wt/wt) of said composition.

In some embodiments, the at least one antiseptic agent is chlorhexidine and its pharmaceutically or veterinarally acceptable salts thereof.

In some embodiments, non-limiting examples of the at least one antibiotic agent includes flouroquinolones, aminoglycosides, ansamycins, carbapanems, cephalosporins, lincosamides, lipopeptides, macrolides, monobactams, nitrofurans, glycopeptides, oxazolidonones, penicillins, penicillin combinations, polypeptides, sulfonamides, tetracyclines, and combinations thereof.

In some embodiments, non-limiting examples of the at least one anti-viral agent includes acyclovir, imiquimod, podofilox, docosanol, penciclovir, podophyllin and combinations thereof.

In some embodiments, at least one topical anti-inflammatory agent can optionally be added to the composition in addition to the at least one antimicrobial agent. Non-limiting examples of anti-inflammatory agents include ibuprofen, diclofenac, felbinac, ketoprofen, peroxicam and combinations thereof.

In some embodiments, the at least one antimicrobial and optionally the at least one anti-inflammatory is topical antimicrobial and topical anti-inflammatory.

In at least one embodiment, the compositions described herein can include sodium hexmetaphosphate.

Where applicable, the at least one antimicrobial and optionally the at least one anti-inflammatory used in the compositions described herein include the use of pharmaceutically or veterinarally acceptable salts thereof.

In some embodiments, the compositions described herein can have a cosmetic use for the prevention or removal of tear and/or salivary stains of an animal's fur, in particular the fur of dogs and cats, resulting from hyper-salivation or self-licking of the fur.

In some embodiments, the compositions described herein can be formulated in forms that are well known in the art. Non-limiting examples of which include washes, flushes, gels, lavages, foams, aqueous solutions, solid edible indigestible animal food products, pastes, patches, sprays, wipes, creams, moistened swab, moistened cotton, moistened gauze, moistened towels, moistened towelettes, polymer foam, medically acceptable sponge, syringe, squeeze bottle, dropper or pipette, and can be administered by one of ordinary skill in the art, such as a veterinarian or a physician, using methods that are well known in the art.

### DEFINITIONS

All technical and scientific terms used herein, unless otherwise defined below, are intended to have the same meaning as commonly understood by one of ordinary skill in the art. References to methods employed herein are intended to refer to the methods as commonly understood in the art, including variations on those methods or substitutions of equivalent methods which would be apparent to one of skill in the art.

As used in this specification, the singular forms “a”, “an” and “the” specifically also encompass the plural forms of the terms to which they refer, unless the content clearly dictates otherwise. For example, reference to “antimicrobial” includes mixtures of antimicrobials.

The term a “therapeutically effective amount” as used herein means an amount of the composition, which when administered according to a desired dosage regimen, is sufficient to at least partially attain the desired therapeutic effect, or delay the onset of, or inhibit the progression of, halt, partially or fully the onset or progression of the infection or is able to reverse or partially reverse the antimicrobial sensitivity of the pathogenic microbe(s).

The term a “preventative effective amount” as used herein means an amount of the composition, which when administered according to a desired dosage regimen, is sufficient to at least partially prevent or delay the onset of the infection.

The term “ophthalmic composition(s)” as used herein denotes a composition intended for application in, around, or near the eye of an animal or human.

The term “ophthalmic diseases” or “ophthalmic disorders” refers to diseases and disorders affecting the eye or in close proximity to the eye.

The term “tear stain(s)” refers to the abnormal pigmentation of an animal's fur, which may be due to excessive tearing of the animal's eye.

The term “salivary stain(s)” refers to the abnormal pigmentation of an animal's fur, which may be due to hypersalivation by the animal or self-licking of the animal's fur coat.

The term “oral diseases” or “oral disorders” refers to diseases and disorders affecting the oral cavity or associated medical conditions. The treatment and/or prevention of oral diseases or disorders using the compositions described herein include, but are not limited to, odontostomatological pathologies, such as for example, treatment of dental extractions, ulcerative stomatitis, granulomas, infections of the oral cavity (e.g., inflammatory states of the vestibule of the mouth and/or gums of animals), formation of plaque and/or tartar, traumatic lesions at the level of the oral cavity, dental caries; and periodontal diseases (e.g., gingivitis, adult periodontitis, early-onset periodontitis, etc.).

The term “oropharyngeal” or oropharyngeal area” as used herein, particularly in relation to equines refers to the upper respiratory tract of equines, i.e., the portion of the respiratory system extending from the nares to the larynx. Some of the anatomic structures in the oropharyngeal area include nasal cavities, paranasal sinuses (frontal and maxillary sinuses), nasopharynx, soft palate, tongue, nasolacrimal duct, and larynx. Accordingly, diseases and disorders of the oropharayngeal area include diseases and disorders of any one or more of some of these anatomic structures.

As used herein, “pharmaceutically active agent” or “veterinarally active agent” shall refer to active agents that can be used for treating ophthalmic disorders, oral cavity disorders in animals or humans, or disorders of the oropharyngeal area and/or guttural pouch in equines. Pharmaceutically or veterinarally active agents include antimicrobial agents (including antiviral agents, antifungal agents and bacteriocidal and bacteriostatic agents). Antiseptics and antibiotics are contemplated as pharmaceutically active agents in light of reported evidence of synergistic effects when using combinations of TRIS-EDTA solutions with such antimicrobials (see for example Blue et al. (1974), Wooley et al. (1983), Farca et al. (1997), see also PCT/US2003/011300 and PCT/US01/29133).

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified throughout this disclosure, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

The various embodiments of the invention described herein may suitably comprise, consist essentially of, or consist of, at least one buffering agent, at least one chelating agent, and at least one antimicrobial. Certain embodiments of the invention may suitably comprise, consist essentially of, or consist of TRIS, EDTA and at least one antiseptic. Certain other embodiments of the invention may suitably comprise, consist essentially of, or consist of TRIS, EDTA and chlorhexidine. Certain embodiments of the invention may suitably comprise, consist essentially of, or consist of, at least one buffering agent, at least one chelating agent, and at least one antimicrobial and optionally at least one anti-inflammatory.

The at least one buffering agent suitable for use in the compositions described herein include any of those which are known in the art, or may hereafter be developed, and which can maintain the pH of the solution in the prescribed range for a substantial period of storage. Non-limiting examples of suitable buffering agents include citrate buffers, phosphate buffers, borate buffers, TRIS(hydroxymethyl)aminomethane (TRIS) buffers, sodium bicarbonate, N,N′-bis(2-hydroxyethyl)glycine (BICIN), 2-[bis(2-hydroxyethyl)amino]-2-(hydroxymethyl)-1,3-propanediol (BISTRIS), 2-(cyclohexylamino)ethane-2-sulfonic acid (CHES), N-2-(hydroxyethyl)piperazine-N′-2-ethanesulfonic acid (HEPES), N-(2-hydroxyethyl)piperazine-N′-3-propanesulfonic acid (HEPPS), morpholinoethanesulfonic acid (MES), morpholinopropanesulfonic acid (MOPS), piperazine-N,N′-bis(2-ethanesulfonic acid) (PIPES), N-TRIS(hydroxymethyl)methyl-2-aminoethanesulfonic acid (TES), N-TRIS(hydroxymethyl)methyl-3-aminopropanesulfonic acid (TAPS), and N-TRIS(hydroxymethyl)methylglycine (TRICINE), and combinations thereof.

In some embodiments the at least one buffering agent is TRIS(hydroxymethyl)aminomethane (TRIS), which is also known as tromethamine or trimethylol aminomethane or 2-amino-2-(hydroxymethyl)-1,3-propane-diol. TRIS buffers are commercially available as, e.g., from Sigma Chemical Co., St. Louis, Mo.

The at least one metal ion chelating agent or chelating agent suitable for use in the compositions described herein include any of those well known in the art, or may hereafter be developed. Non-limiting examples of suitable chelating agent include citric acid, phosphates, the di-, tri- and tetra-sodium salts of ethylene diamine tetraacetic acid (EDTA), ethylene glycol-bis-(b-aminoethylether)-N,N,N′,N′-tetraacetic acid (EGTA); 1,2-bis(2-aminophenoxy)ethane-N,N,N′,N′-tetraacetic acid (BAPTA); ethylene-N,N′-diglycine (EDDA); 2,2′-(ethylendiimino)-dibutyric acid (EBDA); lauroyl EDTA; dilauroyl EDTA, triethylene tetramine dihydrochloride (TRIEN), diethylenetriamin-pentaacetic acid (DPTA), triethylenetetramine hexaacetic acid (TTG), deferoxamine, Dimercaprol, zinc citrate, penicilamine succimer, Editronate, and edetate calcium disodium.

In some embodiments, the at least one chelating agent is a derivative of EDTA. In at least one embodiment, the chelating agent is the disodium salt dihydrate of EDTA (Na₂EDTA.2H₂O). Chelating agents, such as for example EDTA are commercially available as, e.g., from Sigma Chemical Co., St. Louis. Mo.

Most pharmaceutically or veterinarally active agents are either weak acids or bases that form ionized and nonionized species in solution. The nonionized species are usually lipid soluble and are able to penetrate the lipid membrane of a cell whereas the ionized species have difficulty in crossing the cell membrane. At steady state, the acidic species will accumulate on the more basic side of a cell membrane while basic species tend to accumulate on the acidic side of the cell membrane. The alkaline nature of the wash solution of the instant invention should produce an external environment wherein the basic, ionized species of drugs should accumulate to the interior of the cell membrane. This occurs as a purely physical process that does not require active transport. Goodman and Gilman's The Pharmacological Basis of Therapeutics, 10^{th} Ed., Chapt 1., McGraw Hill Companies Inc., New York (2001). The exudates associated with infected lesions are acidic in nature and can inactivate or reduce the efficacy of many commonly used antimicrobial agents. Thus, the pH of the compositions described herein should be optimized to neutralize the acidic environment of the lesion thereby allowing for increased efficacy for the commonly used antimicrobial agents.

Accordingly, the amount of buffering agent in the composition(s) described herein will depend on the nature of the buffering agent and the pH required for the composition(s). Similarly, the amount of the chelating agent present in the composition(s) will depend on the nature of the chelating agent and its effectiveness in chelating ions from a microbe's, particularly bacteria's, cell wall. The chelating agent is included in an amount effective to enhance the effectiveness of the antimicrobial component.

In certain embodiments, the TRIS buffering agent is present in quantities ranging from between 0.001% (wt/wt) to 30% (wt/wt) of said composition. In certain other embodiments the TRIS buffering agent is present in quantities ranging from between 0.01% (wt/wt) to 25% (wt/wt) of said composition. In still other embodiments, the TRIS buffering agent is present in quantities ranging from between 0.05% (wt/wt) to 10% (wt/wt) of said composition.

In some embodiments directed to treating and/or preventing ophthalmic diseases or disorders, the TRIS buffering agent in the compositions is present in quantities ranging from between 0.05% (wt/wt) to 2.5% (wt/wt) of said composition. In certain other embodiments directed to treating and/or preventing ophthalmic diseases or disorders, the TRIS buffering agent is present in quantities ranging from between 0.1% (wt/wt) to 1.5% (wt/wt) of said composition. In still other embodiments directed to treating and/or preventing ophthalmic related diseases or disorders, the TRIS buffering agent is present in quantities ranging from between 0.3% (wt/wt) to 1.0% (wt/wt) of said composition. In at least one embodiment directed to treating and/or preventing ophthalmic related disorders, the TRIS buffering agent is present at 0.45% (wt/wt) of said composition.

In some embodiments directed to treating and/or preventing diseases or disorders of the oral cavity, the TRIS buffering agent in the compositions is present in quantities ranging from between 0.001% (wt/wt) to 30% (wt/wt) of said composition. In certain other embodiments directed to treating and/or preventing diseases or disorders of the oral cavity, the TRIS buffering agent is present in quantities ranging from between 0.01% (wt/wt) to 25% (wt/wt of said composition. In still other embodiments directed to treating and/or preventing diseases or disorders of the oral cavity, the TRIS buffering agent is present in quantities ranging from between 0.05% (wt/wt) to 10% (wt/wt) of said composition. In at least one embodiment directed to treating and/or preventing disease or disorders of the oral cavity, the TRIS buffering agent is present at 0.45% (wt/wt) of said composition.

In some embodiments directed to treating and/or preventing diseases or disorders of the oropharyngeal area and/or guttural pouches in equines, the TRIS buffering agent in the compositions is present in quantities ranging from between 0.001% (wt/wt) to 30% (wt/wt) of said composition. In certain other embodiments directed to treating and/or preventing diseases or disorders of the oropharyngeal area and/or guttural pouches in equines, the TRIS buffering agent is present in quantities ranging from between 0.01% (wt/wt) to 25% (wt/wt) of said composition. In still other embodiments directed to treating and/or preventing diseases or disorders of the oropharyngeal area and/or guttural pouches in equines, the TRIS buffering agent is present in quantities ranging from between 0.05% (wt/wt) to 10% (wt/wt) of said composition. In at least one embodiment directed to treating and/or preventing disease or disorders of the oropharyngeal area and/or guttural pouches in equines, the TRIS buffering agent is present at 0.45% (wt/wt) of said composition

In some embodiments, the EDTA chelating agent and/or a derivative thereof is present in quantities ranging from between 0.001% (wt/wt) to 18% (wt/wt) of said composition. In some other embodiments, the EDTA chelating agent and/or a derivative thereof is present in quantities ranging from between 0.01% (wt/wt) to 10% (wt/wt) of said composition. In still other embodiments, the EDTA chelating agent and/or a derivative thereof is present in quantities ranging from between 0.05% (wt/wt) to 5% (wt/wt) of said composition.

In some embodiments directed to treating and/or preventing ophthalmic diseases or disorders, the EDTA chelating agent and/or a derivative thereof in the compositions is present in quantities ranging from between 0.01% (wt/wt) to 0.5% (wt/wt) of said composition. In certain other embodiments directed to treating and/or preventing ophthalmic diseases or disorders, the EDTA chelating agent and/or a derivative thereof is present in quantities ranging from between 0.1% (wt/wt) to 0.4% (wt/wt of said composition. In still other embodiments directed to treating and/or preventing ophthalmic related diseases or disorders, the EDTA chelating agent and/or a derivative thereof is present in quantities ranging from between 0.2% (wt/wt) to 0.3% (wt/wt) of said composition. In at least one embodiment directed to treating and/or preventing ophthalmic related disorders, the EDTA chelating agent and/or a derivative thereof is present at 0.12% (wt/wt) of said composition.

In some embodiments directed to treating and/or preventing diseases or disorders of the oral cavity, the EDTA chelating agent and/or a derivative thereof in the compositions is present in quantities ranging from between 0.001% (wt/wt) to 18% (wt/wt) of said composition. In certain other embodiments directed to treating and/or preventing diseases or disorders of the oral cavity, the EDTA chelating agent and/or a derivative thereof is present in quantities ranging from between 0.01% (wt/wt) to 10% (wt/wt of said composition. In still other embodiments directed to treating and/or preventing diseases or disorders of the oral cavity, the EDTA chelating agent and/or a derivative thereof is present in quantities ranging from between 0.05% (wt/wt) to 5% (wt/wt) of said composition. In at least one embodiment directed to treating and/or preventing disease or disorders of the oral cavity, the EDTA chelating agent and/or a derivative thereof is present at 0.12% (wt/wt) of said composition.

In some embodiments directed to treating and/or preventing diseases or disorders of the oropharyngeal area and/or guttural pouches in equines, the EDTA chelating agent and/or a derivative thereof in the compositions is present in quantities ranging from between 0.001% (wt/wt) to 18% (wt/wt) of said composition. In certain other embodiments directed to treating and/or preventing diseases or disorders of the oropharyngeal area and/or guttural pouches in equines, the EDTA chelating agent and/or a derivative thereof is present in quantities ranging from between 0.01% (wt/wt) to 10% (wt/wt of said composition. In still other embodiments directed to treating and/or preventing diseases or disorders of the oropharyngeal area and/or guttural pouches in equines, the EDTA chelating agent and/or a derivative thereof is present in quantities ranging from between 0.05% (wt/wt) to 5% (wt/wt) of said composition. In at least one embodiment directed to treating and/or preventing disease or disorders of the oropharyngeal area and/or guttural pouches in equines, the EDTA chelating agent and/or a derivative thereof is present at 0.12% (wt/wt) of said composition

In some embodiments, the buffer solution of the compositions described herein is of the TRIS-EDTA type. It is known in the art that the presence of this type of buffer solution reinforces the action of certain antimicrobial agents, such as for example, chlorhexidine and its derivatives, and antibiotics, thus allowing the use of these antimicrobial agents, with the same treatment efficacy, in lower quantities with respect to compositions based on chlorhexidine and its derivatives, or antibiotics alone.

In some embodiments, the compositions described herein have TRIS-EDTA buffered to a pH ranging from between 5.5 to 8.5, in certain other embodiments, the compositions described herein have TRIS-EDTA buffered to a pH ranging from between 6.0 to 8.0, and in still other embodiments, the compositions described herein have TRIS-EDTA buffered to a pH ranging from between 6.5 to 7.5, to make them suitable for use. In order to do so, a pH adjuster or suitable acid buffer such as HCl or sulphuric acid may be added. Although any pH adjuster may be used, the use of tromethamine HCL or TRIS(hydroxymethyl)aminomethane hydrochloride is preferred. In certain embodiments, the compositions described herein, the ratio of EDTA:pH adjuster:TRIS is approximately 1:1.25:3.875.

As mentioned above, the pH of the compositions described herein will depend on the use of said compositions. For example, in certain embodiments where the compositions are to be used for the treatment and/or prevention of ophthalmic related disorders, the pH of the compositions should be optimized for the ocular environment of the animal or human for which the composition is to be used. Accordingly, in certain embodiments, where compositions are intended for treating and/or preventing ophthalmic diseases or disorders, the TRIS-EDTA should be buffered to a pH ranging from between 6.0 to 8.0, in certain embodiments, the TRIS-EDTA should be buffered to a pH ranging from between 6.8 to 7.8, and in certain other embodiments, the TRIS-EDTA should be buffered to a pH ranging from between 7.3 to 7.7 (the difference in pH of tears between dogs and humans is minimal). In embodiments where the compositions are intended for treating or preventing diseases or disorders related to the oral cavity, the TRIS-EDTA should be buffered to a pH ranging from between 6.5 to 7.5 (the mean salivary pH of dogs is 7.5 and the mean salivary pH of humans is 6.5). In embodiments, where the compositions are to be used for treating and/or preventing oropharyngeal and/or guttural pouch diseases or disorders in equines, the TRIS-EDTA should be buffered to a pH ranging from between 4.0 to 10.0. In certain embodiments, where the compositions are used for treating and/or preventing oropharyngeal and/or guttural pouch diseases or disorders in equines, the TRS-EDTA should be buffered to a pH ranging from 7.5-8.0. In still other embodiments, compositions used for treating and/or preventing oropharyngeal and/or guttural pouch diseases or disorders in equines should have a pH of 8.0.

Certain embodiments of the compositions described herein include at least one antimicrobial to aid in the treatment and/or prevention of the disease or disorder being treated. Non-limiting examples of the at least one antimicrobial agent include at least one antiseptic agent, at least one antifungal agent, at least one antibiotic agent, at least one antiviral agent, at least one antiparasitic agent and combinations thereof.

In certain embodiments, the at least one anti-microbial agent is at least one anti-septic agent. Non-limiting examples of the at least one antiseptic agent include triclosan; iodoform; quaternary ammonium compounds, such as for example, benzalkonium chloride, cetyl trimethylammonium bromide, cetylpyridinium chloride, and benzethonium chloride; and biguanides, such as PHMB, N-alkyl-2-pyrrolidone, chlorhexidine, polyquaternium-1, hexetidine, bronopol, alexidine and combinations thereof.

In certain embodiments, the at least one antiseptic agent is present in quantities ranging from between 0.001% to 5% of said composition, in certain other embodiments, the at least one antiseptic agent is present in quantities ranging between 0.0012%5 to 0.15% of said composition, in still other embodiments, the at least one antiseptic agent is present in quantities ranging from between 0.00125% to 0.005% of said composition, and in some other embodiments, the at least one antiseptic agent is present in quantities ranging from 0.00125% to 0.0025% of said composition. In certain embodiments, the at least one antiseptic agent is chlorhexidine and its pharmaceutically or veterinarally acceptable salts.

In certain other embodiments, the at least one antimicrobial agent is at least one antibiotic agent. Non-limiting examples of the at least one antibiotic agent include flouroquinolones, aminoglycosides, ansamycins, carbapanems, cephalosporins, lincosamides, lipopeptides, macrolides, monobactams, nitrofurans, glycopeptides, oxazolidonones, penicillins, penicillin combinations, polypeptides, sulfonamides, tetracyclines, and combinations thereof.

In some of the embodiments, the at least one antimicrobial agent is at least one antifungal agent. Non-limiting examples of the at least one antifungal agent include butenafine, clotrimazole, ketoconazole, miconazole, nystatin, terbinafine, ciclopirox, econazole, luliconazole, naftifine, oxiconazole, and combinations thereof.

In some embodiments, the at least one antimicrobial agent is at least one antiviral agent. Non-limiting examples of the at least one antiviral agent includes acyclovir, imiquimod, podofilox, docosanol, penciclovir, podophyllin and combinations thereof.

In some embodiments, the at least one antimicrobial agent includes at least one antiparasitic agent. Non-limiting examples of the at least one antiparasitic agent include antinematodes, anticestodes, antitrematodes, antoamoebics, antiprotozoals and combinations thereof. Non-limiting examples of antinematodes include mebendazole, pyrantel pamoate, thiabendazole, diethylcarbamazine, ivermectin and combinations thereof. Non-limiting examples of anticestodes include niclosamide, praziquantel, albendazole and combinations thereof. Non-limiting examples of antitrematodes include praziquantel. Non-limiting examples of antiamoebics include rifampin, amphotericin B and combinations thereof. Non-limiting examples of antiprotozoals include melarsoprol, eflornithine, metronidazole, tinidazole, and miltefosine.

In some embodiments, the at least one antimicrobial agent can be administered with at least one anti-inflammatory agent. Non-limiting examples of the at least one anti-inflammatory agent includes ibuprofen, diclofenac, felbinac, ketoprofen, peroxicam and combinations thereof.

In certain embodiments, the compositions described herein can further include sodium hexmetaphosphate.

In certain embodiments related to the use of the compositions described herein for treating and/or preventing ophthalmic diseases or disorders, the at least one antimicrobial agent is present in quantities ranging from between 0.000001% (wt/wt) to about 5.0% (wt/wt) of said composition. In certain embodiments related to the use of the compositions described herein for treating and/or preventing ophthalmic diseases or disorders, the at least one antimicrobial agent is present at 0.002% (wt/wt) of said composition. In certain embodiments related to the use of the compositions for treating and/or preventing ophthalmic disorders, the at least one antimicrobial agent is at least one antiseptic, wherein the at least one antiseptic is chlorhexidine. In certain embodiments, the tear stains of animals, such as dogs and cats, can be wiped away using compositions described herein wherein the at least one antimicrobial agent is at least one antiseptic, such as for example chlorhexidine, at 0.002% (wt/wt) of said composition.

In certain embodiments related to the use of the compositions described herein for treating and/or preventing oropharyngeal and/or guttural pouch diseases or disorders in equines, the at least one antimicrobial agent is present in quantities ranging from between 0.001% (wt/wt) to about 5% (wt/wt) of said composition. In certain embodiments related to the use of the compositions described herein for treating and/or preventing oropharyngeal and/or guttural pouch diseases or disorders in equines, the at least one antimicrobial agent is present at 0.05% (wt/wt) of said composition. In certain embodiments related to the use of the compositions for treating and/or preventing oropharyngeal and/or guttural pouch diseases or disorders in equines, the at least one antimicrobial agent is at least one antiseptic, wherein the at least one antiseptic is chlorhexidine present at 0.05% (wt/wt) of said composition.

In certain embodiments related to the use of the compositions described herein for treating and/or preventing oral cavity diseases or disorders, the at least one antimicrobial agent is present in quantities ranging from between 0.0001% (wt/wt) to 10% (wt/wt) of said composition. In certain embodiments related to the use of the compositions described herein for treating and/or preventing diseases or disorders related to the oral cavity, the at least one antimicrobial agent is present at 0.12% (wt/wt) of said composition. In certain embodiments related to the use of the compositions for treating and/or preventing diseases or disorders of the oral cavity, the at least one antimicrobial agent is at least one antiseptic, wherein the at least one antiseptic is chlorhexidine present at 0.12% (wt/wt) of said composition.

An attending veterinarian or physician, depending on the severity as well as the general age, health and weight of the subject being treated and type of ophthalmic or oral disorder, can determine the therapeutically effective amount or the preventative effective amount and type of the at least anti-microbial agent, and, if necessary, the at least one anti-inflammatory to be administered to an animal or human suffering from a particular ophthalmic or oral cavity disorder. Similarly, the attending veterinarian can determine the therapeutically effective amount or the preventative effective amount and type of the at least one antimicrobial agent, and if necessary, the at least one anti-inflammatory agent to be administered to an equine suffering from an oropharyngeal and/or guttural pouch disorder. It will also be apparent to a physician or veterinarian that the amount and type of the at least one buffering agent, the at least one chelating agent, as well as the final pH of the compositions described herein, will depend on the organ as well as the type and severity of disease or disorder being treated. For example, the attending physician or veterinarian can first measure the pH of the fluids (tears, saliva or guttural pouch excretion or fluid) of the affected organ, using methods well known in the art, and subsequently adjusted the pH of the compositions described herein so as to return the pH of the organ being treated to its normal physiological pH. As stated above, although any pH adjuster may be used, the use of tromethamine HCL or TRIS(hydroxymethyl)aminomethane hydrochloride is preferred.

The compositions described herein can be formulated in a variety of forms well know in the art. Non-limiting examples of the forms in which the compositions described herein can be formulated as include gels, foams, aqueous solutions, solid edible indigestible animal food products, chewing gum, animal food products, pastes, patches, sprays, wipes, creams, moistened swab, moistened cotton, moistened gauze, moistened towels, moistened towelettes, polymer foam, medically acceptable sponge, syringe, or squeeze bottle.

The above-exemplified forms of the compositions described herein can be manufactured by methods well known to one of skill in the art of formulation science. Additionally, the compositions described herein may include other optional excipients to aid in the manufacturing and/or administration of the compositions described herein. Non-limiting examples of such excipients are well known in the art and include flavaourants, colorants, palatants, antioxidants, viscosity modifying, tonicity agents, drug carriers, sustained-release agents, comfort-enhancing agents, emulsifiers, solubilizing aids, lubricants, binding agents and other stabilizing agents to aid in the manufacturing and/or administration of the compositions.

In certain embodiments, the compositions described herein can be used in the form of aqueous solutions as, for example, a wash, a flush, a lavage, for moistening swabs, cotton pads, gauze, towels, or towelletes. The aqueous solution of the compositions described herein can be made by means well known in the art.

In one embodiment, the compositions described herein may be aqueous and contain 0-90% water. In other embodiments, the aqueous compositions described herein may contain 20-80% water. In still other embodiments, aqueous compositions may contain 50-70% water. The water may further comprise water that is plain, distilled, sterile, demineralized or deionized.

In at least one embodiment, the aqueous solutions of the compositions described herein can be prepared by adding the ingredients as follows. Add the at least one buffering agent to water. Adjust the pH of the solution to the desired pH depending on the organ that is to be treated. Add the at least one chelating agent and the tonicity agent, and optionally any other excipients, such as for example, a tonicity agent, a viscosity-modifying agent, a comfort-enhancing agent. Add the at least one anti-microbial agent and optionally the at least one anti-inflammatory agent. The final product can be rendered sterile, if necessary, by sterile filtration, heat sterilization or a combination thereof.

In certain embodiments, the compositions described herein can be formulated as a dry, solid, water soluble, or dispersible unit dosage form, e.g. tablets, or as a food product for animals or humans using methods that are well known to one of skill in the art of formulation science using standard equipment. One type of tablet would contain the at least one buffering agent, the at least one anti-microbial agent, and the at least one chelating agent. Prior to use, the tablet is hydrated and dissolved in a diluent, e.g., water or physiological saline to form an aqueous solution.

In at least one other embodiment, the tablet would contain the at least one buffering agent and the at least one chelating agent, which can be be dissolved in a diluent, e.g., water or physiological saline prior to use to form an aqueous solution. The at least one anti-microbial agent, and optionally the at least one anti-inflammatory, which can be either in a solid form or aqueous solution, can then be added to the buffered solution.

In at least one embodiment, the compositions described herein can be formulated as a food product for animals or humans.

In at least one embodiment, the compositions described herein can be incorporated into pet food or treat. In another embodiment, the compositions described herein can be coated onto the pet food or treat. To give a more pleasant taste to the composition and facilitate its administration to animals, the compositions can also include one or more aromas, such as for example, meat aromas or extracts, fish aromas or extracts, vegetable aromas or extracts or fruit aromas or extracts.

Some embodiments contemplate the incorporation of the compositions described herein into edible indigestible food products, e.g. chewing gum.

The compositions described herein can be manufactured and distributed in the form of kits for ease of administration by an attending veterinarian or physician.

In at least one embodiment, the kit comprises a tablet, said tablet comprising (a) a pre-determined amount of the at least one buffering agent, the at least one chelating agent and the at least one anti-microbial agent in the form of a compressed tablet, (b) a predetermined volume of a diluent, e.g., water or physiological saline in an appropriately sized container, and (c) an insert providing instructions on how to re-hydrate the tablet in (a) to be administered to an animal or human in need of such administration. The kit can optionally contain at least one additional anti-microbial not incorporated in (a) and/or at least one anti-inflammatory agent. These optional ingredients can be provided in solid or in aqueous form.

In at least one embodiment, the kit comprises (a) a pre-weighed amount of the least one buffering agent, the at least one chelating agent and the at least one anti-microbial in powder or crystalline form, (b) a predetermined volume of a diluent, e.g., water or physiological saline in an appropriately sized container, and (c) an insert providing instructions on how to reconstitute the powder or crystal in (a) to be administered to an animal or human in need of such administration. The kit can optionally contain at least one additional anti-microbial not included in (a) and/or at least one anti-inflammatory agent. The optional ingredients can be provided in solid or aqueous form.

In at least one embodiment, the kit comprises (a) a concentrated aqueous solution of the at least one buffering agent, the at least one chelating agent, and the at least one anti-microbial agent, (b) a container containing an appropriate amount of a diluent, e.g., water or physiological saline, (c) and empty container, and (d) an insert providing instructions on how to dilute the concentrated aqueous solution of (a) to a desired concentration by adding an appropriate volume of the aqueous solution in (a) with an appropriate volume of the diluent in (d) in container (c) to the desired concentration of the composition to be administered. The kit can optionally contain at least one additional anti-microbial agent not included in (a) and/or at least one anti-inflammatory agent. The optional ingredients can be provided in solid or aqueous form.

The aforementioned containers supplied in the kits can be made from HDPE (high density polyethylene), LDPE (low density polyethylene), polypropylene, poly(ethylene terepthalate) and the like. For eye drops, flexible bottles having conventional dispensing tops are especially suitable for use with the aqueous compositions described herein. Where appropriate, the containers can also be glass ampules. For example, the at least one anti-microbial and/or inflammatory can be provided in the kit in solid or aqueous form in a glass ampule.

In some embodiments, the kits can further comprise a means for delivering the compositions to be administered to the area or organ of an animal or human in need of such administration. Non-limiting examples of such means include a dropper, a pipette, a suitable catheter, towels, gauze, and cotton pads.

The compositions described here in can be administered by one of skill in the art, such as for example, a veterinarian or physician by methods well known in the art. For example, in one embodiment, for treating an oropharyngeal and/or guttural pouch disorder, the veterinarian can administer the compositions described herein as a lavage using an appropriate catheter. In another embodiment, for removing tear or salivary stains, said stains can be removed by wetting a cotton pad or gauze with the appropriate composition and wiping off said stains. Alternatively, the tear or salivary stains can be wiped away using pre-wetted packaged cotton pads, gauze or towelletes.

### EXAMPLES

### Example 1

### Ophthalmic Composition for Use as Eye Drops, Tear Stain Wipes or Opthalmic Wash for Equines

This composition is formulated as an aqueous formulation for treatment of ophthalmic diseases or disorders, such as for example, corneal disorders. For such compositions, water comprised 99.43% of said composition, Disodium EDTA is added at 0.12% to the water while stirring until the EDTA was completely dissolved. TRIS is added next at 0.45% until the TRIS is completely dissolved. While still mixing, 0.002% (wt/wt) of chlorhexidine gluconate is added slowly to the TRIS-EDTA solution. If necessary, the composition is pH-balanced with tromethamine HCL to between 6.8-7.2. The composition is subsequently manufactured as eye drops, as tear stain wipes, or as an ophthalmic wash for equines using methods well known in the art.

### Example 2

### Composition for Salivary Stain Removal

This composition is made as described in Example 1, but is formulated as pre-wetted wipes or towelletes comprising EDTA-Tris (0.45% TRIS/0.02%-0.05% EDTA) and chlorhexidine at 0.002% (wt/wt) of said composition.

### Example 3

### Mouth or Dental Wash

This composition is made as described in Example 1, but is formulated as a mouth wash comprising food-grade EDTA-TRIS, 0.12% chlorhexidrine gluconate and 0.05% sodium hexametaphosphate. The pH of this composition is adjusted to between 6.8 to 7.2.

### Example 4

### Food Bars and Bites

This composition is formulated as solid composition for use as pet food for treating and/or preventing an oral disease or disorder. The composition contains the ingredients in the amounts shown in the table below for a 70 lb batch:

| Ingredient | % | Amount (lb) |
|---|---|---|
| Dry/Solid Phase | | |
| Corn Starch | 7.0 | 4.9 |
| Rice Flour | 35.61 | 24.93 |
| Cheese Powder | 2.5 | 1.8 |
| Colored Pet Food | 10.0 | 7.0 |
| Vitamin E Powder | 0.30 | 0.21 |
| Dextrose | 10.0 | 7.0 |
| Carragenan Gum | 1.0 | 0.7 |
| Sorbic Acid | 1.0 | 0.7 |
| Ascorbic Acid | 0.2 | 0.14 |
| Calcium Propionate | 0.3 | 0.21 |
| TRIS-EDTA | | |
| Disodium EDTA | 0.116 | 0.081 |
| Tromethamine | 0.2 | 0.14 |
| PW Water | 1.4 | 1.0 |
| Liquid Phase | | |
| PW Water | 6.01 | 4.2 |
| Corn Syrup | 18.0 | 12.6 |
| Lecithin | 2.0 | 1.4 |
| Glycerin | 3.75 | 2.6 |
| Chlorhexidine | 0.584 | 0.4088 |
| Gluonate | | |
| Safflower Oil | 0.025 | 0.0175 |
| Clove Oil | 0.004 | 0.0031 |
| Total | 100.00 | |

(a) A drum-band heater is applied to the drum of corn syrup and heated to 110° F. This step is started the day before to ensure that the corn syrup is properly heated.

(b) All of the dry/solid phase materials are added in the order listed to a mixer and mixed to homogeneity.

(c) In a separate container, the EDTA, IRIS and 1.4% of the water is mixed to homogeneity.

(d) The remaining water is next added to the mixture in (c).

(e) In a separate container, all of the liquids listed in the Liquid Phase are mixed to homogeneity.

(f) The mixture from (b), (c) (e) is added and mixed to homogeneity.

(g) Once the mixture from (f) is completely mixed to homogeneity, the final mixture is placed on papered trays and allowed to dry and cut into 5 inch pieces, each weighing approximately 2.7 kg wet/2.5 kg dry.

### Example 6

### Oropharyngeal or Guttural Pouch Flush for Equines

This composition is made according to the process described in Example 1. The composition comprises 99.38% (wt/wt) water, 0.12% (wt/wt) disodium EDTA, 0.45% TRIS, and 0.05% (wt/wt) chlorhexidine gluconate. The pH of the composition can then be adjusted to 7.5-8.0.

1. A method of treating or preventing an oropharyngeal and/or guttural pouch infection in an equine in need of such treatment, said method comprising administering a sufficient amount of an aqueous lavage composition to said oropharyngeal area and/or guttural pouch, said lavage composition comprising a therapeutically effective amount of at least one buffering agent, at least one chelating agent, and at least one antimicrobial.

2. The method of claim 1, wherein said at least one buffering agent is selected from the group consisting of citrate buffers, phosphate buffers, borate buffers, TRIS(hydroxymethyl)aminomethane (TRIS) buffers, sodium bicarbonate, N,N′-bis(2-hydroxyethyl)glycine (BICIN), 2-[bis(2-hydroxyethyl)amino]-2-(hydroxymethyl)-1,3-propanediol (BISTRIS), 2-(cyclohexylamino)ethane-2-sulfonic acid (CHES), N-2-(hydroxyethyl)piperazine-N′-2-ethanesulfonic acid (HEPES), N-(2-hydroxyethyl)piperazine-N′-3-propanesulfonic acid (HEPPS), morpholinoethanesulfonic acid (MES), morpholinopropanesulfonic acid (MOPS), piperazine-N,N′-bis(2-ethanesulfonic acid) (PIPES), N-TRIS(hydroxymethyl)methyl-2-aminoethanesulfonic acid (TES), N-TRIS(hydroxymethyl)methyl-3-aminopropanesulfonic acid (TAPS), and N-TRIS(hydroxymethyl)methylglycine (TRICINE), and combinations thereof.

3. The method of claim 1, wherein said at least one buffering agent is TRIS.

4. The method of claim 1, wherein said at least one chelating agent is selected from the group consisting of citric acid, phosphates, the di-, tri- and tetra-sodium salts of ethylene diamine tetraacetic acid (EDTA), ethylene glycol-bis-(b-aminoethylether)-N,N,N′,N′-tetraacetic acid (EGTA); 1,2-bis(2-aminophenoxy)ethane-N,N,N′,N′-tetraacetic acid (BAPTA); ethylene-N,N′-diglycine (EDDA); 2,2′-(ethylendiimino)-dibutyric acid (EBDA); lauroyl EDTA; dilauroyl EDTA, triethylene tetramine dihydrochloride (TRIEN), diethylenetriamin-pentaacetic acid (DPTA), triethylenetetramine hexaacetic acid (TTG), deferoxamine, Dimercaprol, zinc citrate, penicilamine succimer, Editronate, and edetate calcium disodium.

5. The method of claim 1, wherein the said at least one chelating agent is EDTA.

6. The method of claim 1, wherein the at least one antimicrobial is selected from the group consisting of at least one antifungal, at least one antiseptic, at least one antibiotic, at least one antiviral, at least one antiparasitic and combinations thereof.

7. The method of claim 1, wherein the at least one antimicrobial is at least one antiseptic.

8. The method of claim 1, wherein the at least one antimicrobial is a biguanide and pharmaceutically acceptable salts thereof.

9. The method of claim 1, wherein the at least one antimicrobial is chlorhexidine or pharmaceutically acceptable salts thereof.

10. The method of claim 7, wherein the lavage composition further comprises at least one antibiotic present at a concentration that is therapeutically effective.

11. The method of claim 10, wherein the at least one antibiotic is selected from the group consisting of flouroquinolones, aminoglycosides, ansamycins, carbapanems, cephalosporins, lincosamides, lipopeptides, macrolides, monobactams, nitrofurans, glycopeptides, oxazolidonones, penicillins, penicillin combinations, polypeptides, sulfonamides, tetracyclines, and combinations thereof.

12. The method of claim 1, wherein the lavage composition further comprises at least one topical non-steroidal anti-inflammatory agent present at a concentration that is therapeutically effective.

13. The method of claim 12, wherein the at least one topical non-steroidal anti-inflammatory is selected from the group consisting of ibuprofen, diclofenac, felbinac, ketoprofen, peroxicam and combinations thereof.

14. The method of claim 1, wherein the aqueous lavage composition has a pH of from about 6.5 to about 7.5.

15. The method of claim 1 wherein the at least one buffering agent and the at least one chelating agent is present at a concentration of from about 50% (wt/wt) to about 65% (wt/wt) of said lavage composition.

16. The method of claim 1, wherein the at least one antimicrobial is present at a concentration that is therapeutically effective.

17. The method of claim 9, wherein the chlorhexidine is present at a concentration of from about 0.001% (wt/wt) to about 5% (wt/wt) of said lavage composition.

18. The method of claim 9, wherein the chlorhexidine and pharmaceutically acceptable salts thereof is present at a concentration of about 0.05% (wt/wt) of said lavage composition.

19. A method of treating or preventing disorders relating to the oral cavity of animals comprising administering to an animal in need of such administration a composition consisting essentially of at least one buffering agent, at least one chelating agent and at least one antimicrobial.

20. The method of claim 19, wherein the oral cavity disorder is selected from the group consisting of dental extractions, ulcerative stomatitis, granulomas, periodontitis, infections of the oral cavity, plaque, tartar, traumatic lesions at the level of the oral cavity and combinations thereof.

21. The method of claim 19 wherein said at least one buffering agent is selected from the group consisting of citrate buffers, phosphate buffers, borate buffers, TRIS(hydroxymethyl)aminomethane (TRIS) buffers, sodium bicarbonate, N,N′-bis(2-hydroxyethyl)glycine (BICIN), 2-[bis(2-hydroxyethyl)amino]-2-(hydroxymethyl)-1,3-propanediol (BISTRIS), 2-(cyclohexylamino)ethane-2-sulfonic acid (CHES), N-2-(hydroxyethyl)piperazine-N′-2-ethanesulfonic acid (HEPES), N-(2-hydroxyethyl)piperazine-N′-3-propanesulfonic acid (HEPPS), morpholinoethanesulfonic acid (MES), morpholinopropanesulfonic acid (MOPS), piperazine-N,N′-bis(2-ethanesulfonic acid) (PIPES), N-TRIS(hydroxymethyl)methyl-2-aminoethanesulfonic acid (TES), N-TRIS(hydroxymethyl)methyl-3-aminopropanesulfonic acid (TAPS), and N-TRIS(hydroxymethyl)methylglycine (TRICINE), and combinations thereof.

22. The method of claim 19, wherein said at least one buffering agent is TRIS.

23. The method of claim 19, wherein said at least one chelating agent is selected from the group consisting of citric acid, phosphates, the di-, tri- and tetra-sodium salts of ethylene diamine tetraacetic acid (EDTA), ethylene glycol-bis-(b-aminoethylether)-N,N,N′,N′-tetraacetic acid (EGTA); 1,2-bis(2-aminophenoxy)ethane-N,N,N′,N′-tetraacetic acid (BAPTA); ethylene-N,N′-diglycine (EDDA); 2,2′-(ethylendiimino)-dibutyric acid (EBDA); lauroyl EDTA; dilauroyl EDTA, triethylene tetramine dihydrochloride (TRIEN), diethylenetriamin-pentaacetic acid (DPTA), triethylenetetramine hexaacetic acid (TTG), deferoxamine, Dimercaprol, zinc citrate, penicilamine succimer, Editronate, and edetate calcium disodium.

24. The method of claim 19, wherein the said at least one chelating agent is EDTA.

25. The method of claim 19, wherein said at least one antimicrobial is selected from the group consisting of at least one antifungal, at least one antiseptic, at least one antibiotic, at least one antiviral, and combinations thereof.

26. The method of claim 19, wherein the at least one antimicrobial is at least one antiseptic.

27. The method of claim 19, wherein the at least one antimicrobial is a biguanide and pharmaceutically acceptable salts thereof.

28. The method of claim 19, wherein the at least one antimicrobial is chlorhexidine or pharmaceutically acceptable salts thereof.

29. The method of claim 28, wherein the composition further comprises at least one antibiotic present at a concentration that is therapeutically effective.

30. The method of claim 29, wherein the at least one antibiotic is selected from the group consisting of flouroquinolones, aminoglycosides, ansamycins, carbapanems, cephalosporins, lincosamides, lipopeptides, macrolides, monobactams, nitrofurans, glycopeptides, oxazolidonones, penicillins, penicillin combinations, polypeptides, sulfonamides, tetracyclines, and combinations thereof.

31. The method of claim 19, wherein the composition further comprises at least one topical non-steroidal anti-inflammatory agent present at a concentration that is therapeutically effective.

32. The method of claim 31, wherein the at least one topical non-steroidal anti-inflammatory is selected from the group consisting of ibuprofen, diclofenac, felbinac, ketoprofen, peroxicam and combinations thereof.

33. The method of claim 19, wherein the composition has a pH of from about 7.5 to about 8.5.

34. The method of claim 28, wherein the amount of chlorhexidine is present at a concentration of from about 0.0001% to about 10% (wt/wt) of said composition.

35. The method of claim 28, wherein the amount of chlorhexidine is about 0.12% (wt/wt) of said composition.

36. The method of claim 19, wherein the at least one buffering agent and the at least one chelating agent is present from about 0.002% to about 40% (wt/wt) of said composition.

37. The method of claim 19, wherein said composition is formulated as a solid edible indigestible veterinary food product.

38. The method of claim 19, wherein said composition is coated onto a solid edible indigestible veterinary food product.

39. The method of claim 19, wherein said composition is formulated as a gel, wash, foam, spray, patch, wipe, cream, or toothpaste.

40. A method of removing tear or salivary stains on an animal comprising removing said stains with a composition consisting essentially of at least one buffering agent, at least one chelating agent, and at least one antimicrobial.

41. The method of claim 40 wherein said at least one buffering agent is selected from the group consisting of citrate buffers, phosphate buffers, borate buffers, TRIS(hydroxymethyl)aminomethane (TRIS) buffers, sodium bicarbonate, N,N′-bis(2-hydroxyethyl)glycine (BICIN), 2-[bis(2-hydroxyethyl)amino]-2-(hydroxymethyl)-1,3-propanediol (BISTRIS), 2-(cyclohexylamino)ethane-2-sulfonic acid (CHES), N-2-(hydroxyethyl)piperazine-N′-2-ethanesulfonic acid (HEPES), N-(2-hydroxyethyl)piperazine-N′-3-propanesulfonic acid (HEPPS), morpholinoethanesulfonic acid (MES), morpholinopropanesulfonic acid (MOPS), piperazine-N,N′-bis(2-ethanesulfonic acid) (PIPES), N-TRIS(hydroxymethyl)methyl-2-aminoethanesulfonic acid (TES), N-TRIS(hydroxymethyl)methyl-3-aminopropanesulfonic acid (TAPS), and N-TRIS(hydroxymethyl)methylglycine (TRICINE), and combinations thereof.

42. The method of claim 40, wherein said at least one buffering agent is TRIS.

43. The method of claim 40, wherein said at least one chelating agent is selected from the group consisting of citric acid, phosphates, the di-, tri- and tetra-sodium salts of ethylene diamine tetraacetic acid (EDTA), ethylene glycol-bis-(b-aminoethylether)-N,N,N′,N′-tetraacetic acid (EGTA); 1,2-bis(2-aminophenoxy)ethane-N,N,N′,N′-tetraacetic acid (BAPTA); ethylene-N,N′-diglycine (EDDA); 2,2′-(ethylendiimino)-dibutyric acid (EBDA); lauroyl EDTA; dilauroyl EDTA, triethylene tetramine dihydrochloride (TRIEN), diethylenetriamin-pentaacetic acid (DPTA), triethylenetetramine hexaacetic acid (TTG), deferoxamine, Dimercaprol, zinc citrate, penicilamine succimer, Editronate, and edetate calcium disodium.

44. The method of claim 40, wherein the said at least one chelating agent is EDTA.

45. The method of claim 40, wherein said at least one antimicrobial is selected from the group consisting of at least one antifungal, at least one antiseptic, at least one antibiotic, at least one antiviral, and combinations thereof.

46. The method of claim 40, wherein the at least one antimicrobial is at least one antiseptic.

47. The method of claim 40, wherein the at least one antimicrobial is a biguanide and pharmaceutically acceptable salts thereof.

48. The method of claim 40, wherein the at least one antimicrobial is chlorhexidine or pharmaceutically acceptable salts thereof.

49. The method of claim 48, wherein the composition further comprises at least one antibiotic present at a concentration that is therapeutically effective.

50. The method of claim 49, wherein the at least one antibiotic is selected from the group consisting of flouroquinolones, aminoglycosides, ansamycins, carbapanems, cephalosporins, lincosamides, lipopeptides, macrolides, monobactams, nitrofurans, glycopeptides, oxazolidonones, penicillins, penicillin combinations, polypeptides, sulfonamides, tetracyclines, and combinations thereof.

51. The method of claim 40, wherein the composition further comprises at least one topical non-steroidal anti-inflammatory agent present at a concentration that is therapeutically effective.

52. The method of claim 51, wherein the at least one topical non-steroidal anti-inflammatory is selected from the group consisting of ibuprofen, diclofenac, felbinac, ketoprofen, peroxicam and combinations thereof.

53. The method of claim 40, wherein the composition has a pH of from about 6.5 to about 7.5.

54. The method of claim 48, wherein the amount of chlorhexidine is present at a concentration of from about 0.000001% to about 5.0% (wt/wt) of said composition.

55. The method of claim 48, wherein the amount of chlorhexidine is about 0.002% (wt/wt) of said composition.

56. The method of claim 40, wherein the at least one buffering agent and the at least one chelating agent is present from about 0.002% to about 40% (wt/wt) of said composition.

57. The method of claim 40, wherein the composition is in aqueous solution.

58. The method of claim 40, when the composition is administered to an animal in need of such administration by a moistened swab, moistened cotton, moistened gauze, polymer foam, medically acceptable sponge or other cloth, syringe, squeeze bottle, dropper or pipette.
